(19) United States
(12) Patent Application Publication
(10) Pub. No.: US 2023/0149708 A1
(43) Pub. Date: May 18, 2023

(54) TUMOR TREATING FIELDS (TTF) FOR CANCER TREATMENT

(71) Applicants: UNIVERSITY OF SOUTH CAROLINA, COLUMBIA, SC (US); PRISMA HEALTH - UPSTATE, GREENVILLE, SC (US)

(72) Inventors: JOHN O'CONNELL, GREENVILLE, SC (US); JASON HENDERSON, GREENVILLE, SC (US); JONATHAN STENBECK, GREENVILLE, SC (US)

(21) Appl. No.: 17/985,605

(22) Filed: Nov. 11, 2022

Related U.S. Application Data
(60) Provisional application No. 63/278,704, filed on Nov. 12, 2021.

(51) Int. Cl. A61N 1/36

(52) U.S. Cl. CPC A61N 1/36002; A61N 1/3603

(57) ABSTRACT

The disclosure deals with methodologies and systems for oscillating electric fields (OEF), which can disrupt a cell's ability to divide. Passing these electric fields through, for example, a person's brain (or other anatomical organ or region of the body) possesses the ability to stop cancer cells from growing in patients where disease is expected. These devices can be worn by patients going through treatment to inhibit metastatic disease and to even enhance the sensitivity of established cancer cells to other therapies. Presently disclosed methodologies relate to varying frequency and/or amplitude of the oscillating electric signal for improved treatment effectiveness.

### PRIORITY CLAIM

The present application claims the benefit of priority of U.S. Provisional Patent Application No. 63/278,704, titled “Tumor Treating Fields (TTF) for Cancer Treatment,” filed Nov. 12, 2021, which is fully incorporated herein by reference for all purposes.

### BACKGROUND OF THE PRESENTLY DISCLOSED SUBJECT MATTER

The disclosure deals with a system and method for Tumor Treating Fields (TTF) for cancer and/or other treatments. In one exemplary embodiment disclosed herewith, system and method relate to oscillating electric fields (OEF), which can disrupt a cell's ability to divide. Passing these electric fields through, for example, a person's brain, possesses the ability to stop cancer cells from growing in patients where disease is expected. These devices can be worn by patients going through treatment to inhibit metastatic disease and to even enhance the sensitivity of established cancer cells to other therapies.

Thus far, studies of OEF in different cancer cell lines have nearly exclusively attempted to determine the single frequency of OEF at which maximal cell death is achieved.

TTF represent a very narrow range of OEF as TTF are defined with an upper limit of electric field strength of 2 V/cm^{[3]}. Since commercially available devices for laboratory, animal, and human use barely allow for peak electric field strengths outside the range of TTF, they do not allow for real-time varying of the peak electric field strengths nor the frequency of the current during delivery. It is clear these field strength and frequency limitations are not adequate for research intended to improve these quite primitive capabilities.

Prior art describes systems and methods relating to the treatment of cancer and/or tumors including, but not limited to, U.S. Pat. No. 9,910,453, and US Publication Nos. 2017/0215939, 2017/0120041, and 2019/0308016, the disclosures of which are fully incorporated herein and for all purposes.

### SUMMARY OF THE PRESENTLY DISCLOSED SUBJECT MATTER

The presently disclosed computer system and corresponding and/or associated methodology deals with systems and methods for improved TTF for cancer treatment.

We anticipate that in different phases of cell growth, in three-dimensional (3D) growth models and in-vivo due to the heterogeneity in distribution of cell size and ploidy, the range of dipole moments of the cancer cells would also be heterogeneous. Thus, while a single frequency may be ideal for the average dipole moment in a distribution of cells, we propose a more effective strategy would be to modulate the frequency of the carrier alternating current over a range of frequencies. Furthermore, it has been observed that as cells adapt to a continuous delivery of OEF, likely due to change in size and ploidy of the adapting cells, the ideal frequency of OEF changes over time. Thus, we posit that cells may be less able to adapt to OEF delivered with a spectrum of frequencies rather than a single continuous frequency.

It is to be understood that the presently disclosed subject matter equally relates to associated and/or corresponding methodologies. One exemplary such method relates to methodology for using oscillating electric fields (OEF) for passing through a target volume as treatment. Such methodology preferably may comprise selectively and controllably generating an oscillating electric signal at at least one output thereof; connecting the output to an electrode associated with a target volume; controlling generation of the oscillating electric signal such that the electric field strength output at the electrode is in a range of from 0 up to 10 V/cm and such that the electric field passes through the target volume; and changing at least one of the frequency or amplitude of the oscillating electric signal at least once during a treatment.

Another exemplary such method relates to using TTF for cancer treatment by passing OEF through a targeted anatomical volume of a patient to disrupt a targeted cell's ability to divide as disease treatment. Such methodology preferably includes providing a controllable oscillating electric signal generator, connected to a plurality of electrodes associated with a patient, with the output of the electrodes limited for electric field strength up to 10 V/cm; and controlling at least one of either the frequency or amplitude of the oscillating electric signal to be varied during the course of a treatment.

Other example aspects of the present disclosure are directed to systems, apparatus, tangible, non-transitory computer-readable media, user interfaces, memory devices, and electronic devices for ultra-fast photovoltaic spectroscopy. To implement methodology and technology herewith, one or more processors may be provided and programmed to perform the steps and functions as called for by the presently disclosed subject matter, as will be understood by those of ordinary skill in the art.

Another exemplary embodiment of presently disclosed subject matter relates to apparatus for passing OEF through a target volume as treatment. Such apparatus preferably includes a controllable oscillator for generating an oscillating electric signal at at least one output thereof; at least one electrode connectable with the at least one output, associated with a target volume; a power amplifier for controllably amplifying the oscillating electrical signal at the at least one output, so that the electric field strength output at the electrode when connected to the output is in a range of from 0 to 10 V/cm, and passes through the target volume; and one or more processors programmed to control the output level of the power amplifier, and to control and change at least one of the frequency or amplitude of the oscillating electric signal at least once during a treatment.

Yet another exemplary embodiment of presently disclosed subject matter relates to an apparatus for passing OEF through a targeted anatomical volume of a patient as disease treatment. Such apparatus preferably includes a controllable oscillator for generating an oscillating electric signal at at least one output thereof; at least one electrode connectable with the at least one output, to be associated with a patient; a power amplifier for controllably amplifying the oscillating electrical signal at the at least one output, so that the electric field strength output at the electrode when connected to the output is in a range of from 0 up to 10 V/cm; and one or more processors programmed to control the output level of the power amplifier, and to control and change at least one of the frequency or amplitude of the oscillating electric signal at least once during a patient treatment.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding methodologies as associated with practice of any of the present exemplary devices, and vice versa.

These and other features, aspects and advantages of various embodiments will become better understood with reference to the following description and appended claims. The accompanying figures, which are incorporated in and constitute a part of this specification, illustrate embodiments of the present disclosure and, together with the description, serve to explain the related principles.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1A illustrates a block diagram of a prior art TTF exposure setup;

FIG. 1B illustrates a block diagram of an exemplary presently disclosed TTF exposure setup;

FIG. 2A illustrates an exemplary bench set-up arrangement for generating and delivering OEF to a load, such as multiple electrodes, in accordance with presently disclosed subject matter;

FIG. 2B illustrates an exemplary arrangement of electrodes embedded in a lid of a 6-well cell culture plate, to be powered by the arrangement of FIG. 2A;

FIG. 3A illustrates representative images of MDA-MB-231 TNBC cells at Time 0 (i.e., before any treatment);

FIG. 3B illustrates representative images of MDA-MB-231 TNBC cells after 2 hours of OEF treatment;

FIGS. 3C and 3D illustrate representative images of untreated MDA-MB-231 TNBC cells after 24 hours and 40 hours, respectively; and

FIGS. 3E and 3F illustrate representative images of MDA-MB-231 TNBC cells after OEF exposure for 24 hours and 40 hours, respectively.

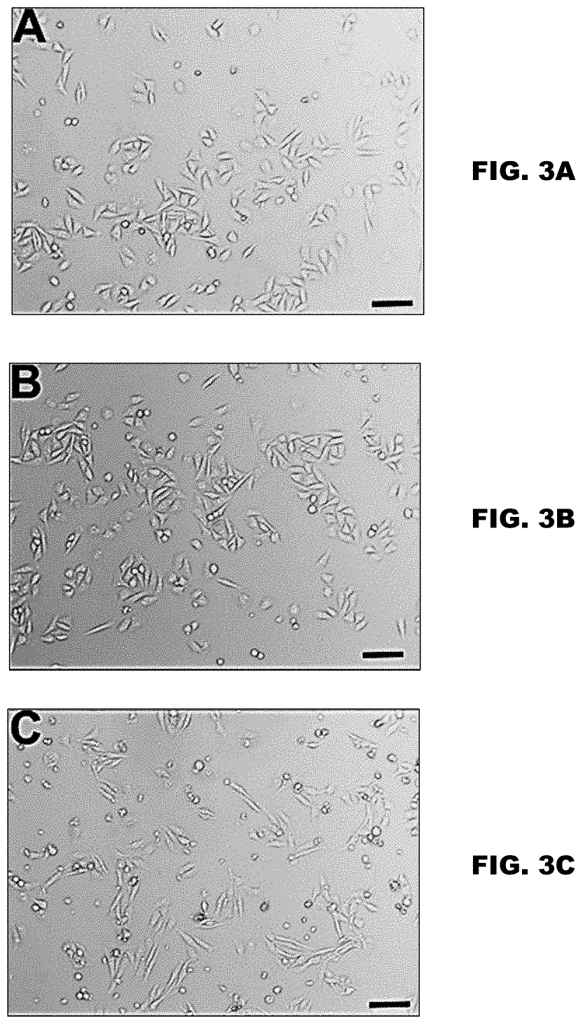

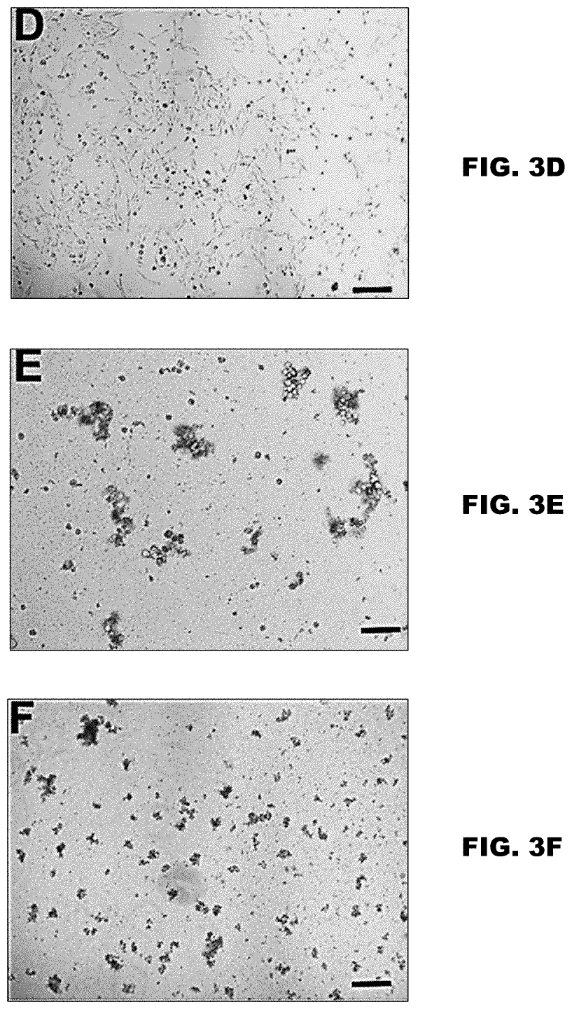

Scale bars for FIGS. 3A, 3B, 3C, 3E, and 3F=50 μm, and for FIG. 3D=100 μm.

Repeat use of reference characters in the present specification and figures is intended to represent the same or analogous features, elements, or steps of the presently disclosed subject matter.

### DETAILED DESCRIPTION OF THE PRESENTLY DISCLOSED SUBJECT MATTER

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment may be used in another embodiment to yield a still further embodiment. Thus, it is intended that the presently disclosed subject matter covers such modifications and variations as come within the scope of the appended claims and their equivalents.

In general, the present disclosure is directed to a system and method which relate to OEF useful for disrupting a targeted cell's ability to divide.

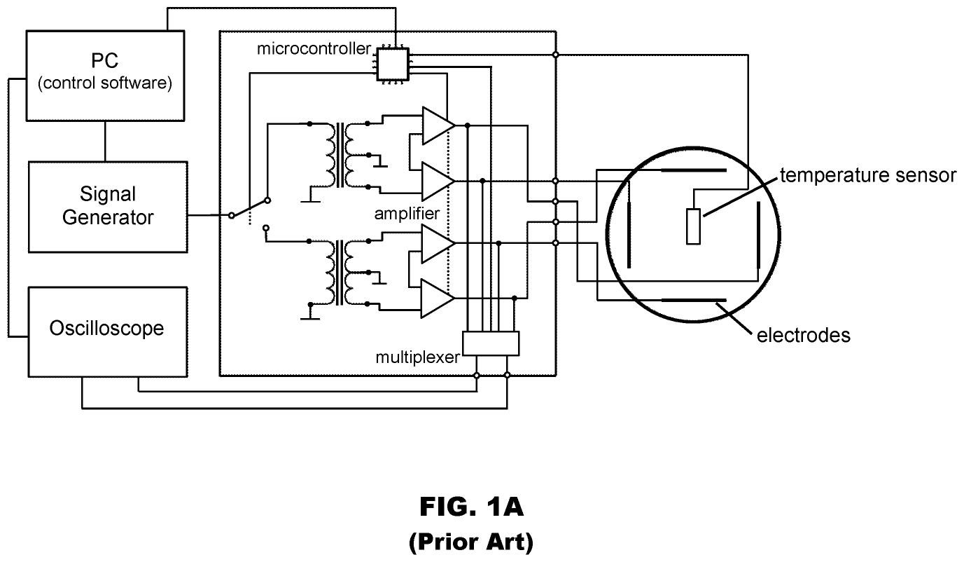

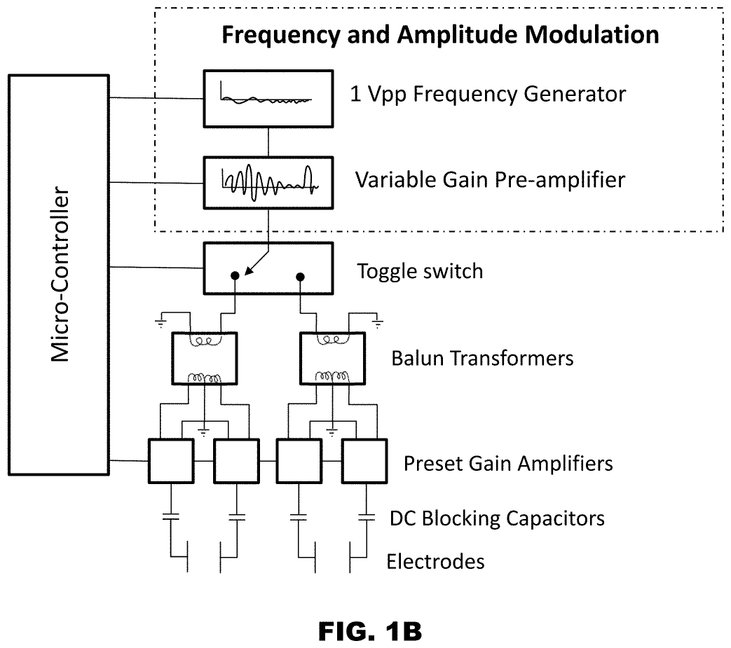

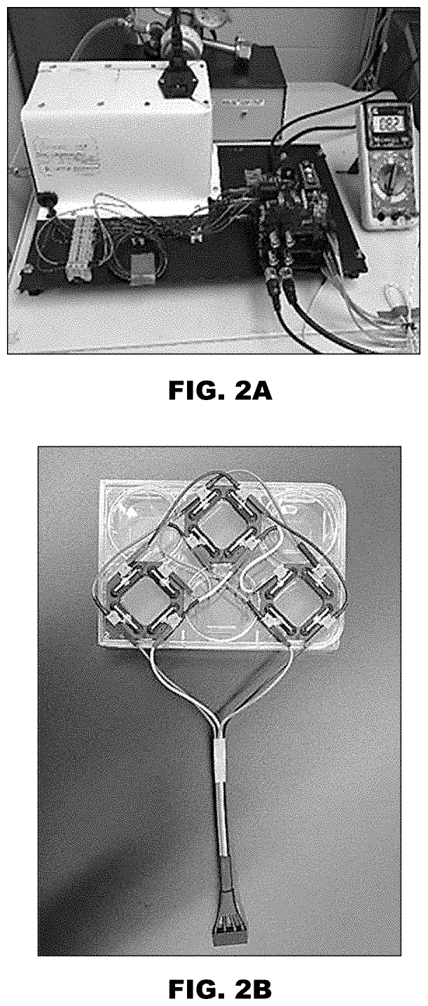

We have designed, built, and tested our own device capable of delivering OEF over a much broader range of electric field intensities. Our device (FIG. 2A) also allows us to test an unlimited number of combinations of alternating current waveforms rather than being restricted to a single frequency or a constant peak voltage. Two other labs in the world outside the Novocure® group have designed their own in-house device capable of delivering OEF to tissue cultures^{[1,2]}. Our device was developed based on the design published by Berkelmann, et al.^{[1]} A block diagram of such prior art device is shown in FIG. 1A of this application. However, our preliminary data goes well beyond the theoretical modeling and testing of commutation times that they recently published.

FIG. 1B illustrates a block diagram representative of the device we have developed. FIG. 2A illustrates an exemplary bench setup arrangement for generating and delivering OEF to a load, such as multiple electrodes, in accordance with presently disclosed subject matter. FIG. 2B illustrates an exemplary arrangement of electrodes embedded in a lid of a 6-well cell culture plate to be powered by the arrangement of FIG. 2A.

TTF represent a very narrow range of OEF as TTF are defined with an upper limit of electric field strength of 2 V/cm^{[3]}. The choice of this upper limit is unclear and seems arbitrary. In fact, studies in rabbits demonstrated electric field intensities as high as 10 V/cm can safely be delivered^{[4]}.

Thus far, studies of OEF in different cancer cell lines have nearly exclusively attempted to determine the single frequency of OEF at which maximal cell death is achieved. We anticipate that in different phases of cell growth—3D growth models and in-vivo—due to the heterogeneity in distribution of cell size and ploidy, the range of dipole moments of the cancer cells would also be heterogeneous. Thus, while a single frequency may be ideal for the average dipole moment in a distribution of cells, we propose a more effective strategy is to modulate the frequency of the carrier, alternating current over a range of frequencies. Furthermore, it has been observed that as cells adapt to a continuous delivery of OEF, likely due to change in size and ploidy of the adapting cells, the ideal frequency of OEF changes over time^{[5]}. Thus, we posit that cells may be less able to adapt to OEF delivered with a spectrum of frequencies rather than a single continuous frequency.

As will be understood by those of ordinary skill in the art, the representative block diagram of an exemplary presently disclosed device in present FIG. 1B we have developed illustrates that a micro-controller can be used to variously control different features of the presently disclosed device in order to achieve desired effects with associated electrode(s). For example and as will be understood, the represented micro-controller, in practice, may comprise one or more processors programmed to: (1) control the output level of the power amplifier (to control and change the frequency of the oscillating electric signal at least once during a patient treatment); (2) modulate the frequency of the oscillating electric signal over a range of frequencies up to GHz range; (3) vary the electric field intensity (“strength” in this context) output at the electrode in a range of from 2 V/cm up to 10 V/cm; and (4) oscillate delivery of current between individual arrangements of electrodes to treat portions of a target volume in accordance with determined custom 3D shaping of electric field distributions. This is similar to the rotating delivery of radiation with modulation of the strength of radiation to a target volume but with the current rotated electronically among electrodes.

One exemplary embodiment may make use of a direct digital synthesis (DDS) method of producing an analog waveform (or equivalent thereto; for example, such as a sine wave) by generating a time-varying signal in digital form and then performing a digital-to-analog conversion. One specific example is the AD9850, which is a highly integrated device that uses advanced DDS technology coupled with an internal high-speed, high-performance, and D/A conversion, which may operate with a frequency range of 0 to 40 MHz. One exemplary variable amplifier which may be utilized is HMC625BCP5EHCP variable amplifier (HMC625BCP5EHCP_31_1_M), which may provide −13.5 to +18 dB gain control in 0.5 dB steps.

As represented by FIG. 2B, such devices (and others in the embodiment) may be controlled by the representative micro-controller, for desired effect. Such approach also supports optional further input from an external function generator, if desired. Similarly, as an option, an output to an oscilloscope may be utilized for monitoring.

Regarding desired effects, as more broadly understood from the complete disclosure herewith, the frequency or amplitude in some embodiments will be changed at least once, which is intended to technically encompass every derivative scenario (i.e., those involving changing more times or more frequently). In some presently disclosed embodiments, however, the frequency/amplitude may be varied with a prescribed profile, such as sawtooth, triangle, sinusoidal, sin\hat{ }2, bimodal (to target two different cell sizes, for example), or other arbitrary modulation shape. Each profile will have a central frequency or amplitude (or multiple peaks each, such as in a bimodal profile) with a max deviation or a weighted deviation to spend more time at specific values or range of values. These could be programmed into an external function generator, or one could utilize an onboard frequency generator to fit any arbitrary signal shape. The shape can be a mathematical function, such as a triangle wave, or can utilize feedback (such as impedance through the electrodes) to dynamically control the frequency or amplitude.

Those of ordinary skill in the art should understand from the complete disclosure herewith that the presently disclosed methodology and system features can alter the frequency or amplitude in single steps (at least once during treatment as currently written) or can vary continuously to maximize effectiveness.

FIG. 3A illustrates representative images of MDA-MB-231 TNBC cells at Time 0 (i.e., before any treatment), while FIG. 3B illustrates representative images of MDA-MB-231 TNBC cells after 2 hours of OEF treatment.

FIGS. 3C and 3D illustrate representative images of untreated MDA-MB-231 TNBC cells after 24 hours and 40 hours, respectively. FIGS. 3E and 3F illustrate representative images of MDA-MB-231 TNBC cells after OEF exposure for 24 hours and 40 hours, respectively.

Scale bars for FIGS. 3A, 3B, 3C, 3E, and 3F=50 μm, and for FIG. 3D=100 μm.

With the delivery of OEF at a constant peak voltage, it has been demonstrated that the vast majority of cells undergo an aberrant mitotic exit.^{[6]} As in the case of spindle poisons, which trigger the spindle assembly checkpoint (SAC), cells affected by OEF exhibit different fates, including death in anaphase or an aberrant exit from mitosis similar to mitotic slippage.^{[7]} Since one mechanism of cancer cells treated with OEF avoids death by exiting mitosis at the transition from metaphase to anaphase, it stands to reason that lethality from OEF would be increased if exit from mitosis could be delayed and a greater percentage of cells progressed into anaphase where death from OEF appears to occur. Therefore, we hypothesize that if the peak voltage of the OEF is everchanging, cellular adaptation to OEF would much more likely be languid and floundering, creating the potential for greater lethality from OEF.

Some presently disclosed alternatives of the foregoing broader concepts include, but are limited to:

Pulses—To synchronize division. Alternatively, synchronize mitotic cycle through small radiation doses or chemical blockers prior to applying TTF, maximizing initial effectiveness of the treatment. Coordinating cell cycle with amplitude modulation, frequency modulation, or commutation time.

Use with other Therapies—Alternatively, the present application of OEF therapy could be used concurrently, sequentially, or both, with at least one other or more of other therapeutic applications. These may include, but are not limited to, therapeutic radiation in single or multiple fractions, ionizing radiation, chemotherapy, immunotherapy, molecular targeted therapy, nanotherapy, surgery, ultrasound, thermal therapy, hyperthermia, hyperbaric oxygen therapy, angiogenesis inhibitors, and antioxidants.

Cream on skin tissue—Placed under electrodes. Use of cream on skin tissue, placed under electrodes to slow skin growth at the site, further limits which cells are undergoing division to those that are cancerous, and thus, permits electrical parameters with higher lethality toward tumor cells (similar to what is used for treating psoriasis or other skin conditions). This could be n-acetyl cysteine (NAC), or vitamin A derivatives, such as Tazarotene, in a cream, gel, or foam (e.g., Avage®, Fabior®, Tazorac®). The cream will also function as a dielectric gel between the electrodes and skin as is currently used.

Cream on skin tissue—Penetrates Skin. Use of cream on skin tissue that penetrates into the skin alters its electrical impedance toward a more favorable value, minimizing the skin's influence on the electric field's shape and strength. This can be customized for each patient's skin to normalize the conditions (bioimpedance) between patients, assisting in simplifying the treatment planning steps as this may also reduce simulation computation time. Zylö Therapeutics, Inc. has a drug delivery product that might work as the transport mechanism for this.

Rotate electric field. Use of 3D shaping and rotation of the electric field to address more than the simple 2 orthogonal orientations.

Since commercially available devices for laboratory, animal, and human use barely allow for peak electric field strengths outside the range of TTF, they do not allow for real-time varying of the peak electric field strengths or the frequency of the current during delivery. It is clear that they are not adequate for research intended to improve on their quite primitive capabilities.

Furthermore, the present method for delivering OEF is to position large pairs of electrodes or electrode arrays on opposite sides of their intended targets with one pair positioned orthogonal to the other pair and alternating the delivery of current between the pairs. This results in delivering OEF to nearly the entire volume of tissue between electrodes. We propose designing an array of much smaller and more numerous electrodes to which current can be delivered individually and at significantly higher frequencies (e.g., the MHz or even GHz range). With such a design, by oscillating delivery of current between individual arrangements of electrodes to treat portions of the target volume very rapidly, custom 3D shaping of electric field distributions at much lower frequencies (i.e., 100 to 500 kHz) could be achieved. This is akin to a rotating delivery of radiation to modulate the strength of radiation to a target volume except that the current would be rotated among electrodes electronically and not mechanically, which would greatly reduce the total current at any single point on the skin and would achieve far higher field strengths than presently attainable.

We intend to optimize the choice of materials used in the electrode design and to evaluate the possible need for real-time cooling of the electrodes during OEF delivery in order to eliminate the risk of thermal damage to the skin. For example, some embodiments may involve the use of Litz wire, which is a particular type of woven wire or cable used in electronics to carry alternating current (AC) at radio frequencies. The design of Litz wire is such that it reduces skin effects (of the wire) and proximity effect losses in conductors used at frequencies up to about 1 MHz, and typically may include many thin wire strands, individually insulated and twisted or woven together. Litz wire 40/40 (40 strands of 40 gauge individually insulated wire) may be used, for example, in some presently disclosed exemplary electrode embodiments, to reduce the “skin effect”—carrying AC with minimum resistance at presently disclosed frequency ranges. Some examples may be optimized for such as 100 to 200 kHz. For an example optimized for 200 KHZ to 350 KHZ, electrodes might preferably use 42 AWG. In general, for some applications, 40 gauge is sufficiently small resistance without the need to have even smaller wires, especially when more wires are included in the bundle. Operation of the presently disclosed technology can, in some instances, result in electrical current demands only requiring about five strands, though larger numbers, such as forty may be used.

Since the mechanism of cellular damage on the skin from OEF appears similar to spindle poisons such as Taxol®, it stands to reason that something that protects against cell death from Taxol® may also protect against cell death from OEF.

Some experimental results achieved to date with the presently disclosed technology are very encouraging. With use of frequency modulation as presently disclosed herewith in conjunction with test/study cell setups using electrodes associated with a single or multi-well cell culture plate, we have found that after 72 hours of treatment, none of the breast cancer cells remained and normal breast cells continued growing. Delivering OEF with or without frequency modulation, we have found a significant percentage of cancer cell death after 24 hours. By 48 hours, there was some rebound in the population of breast cancer cells with or without frequency modulation. However, at 72 hours, the population of breast cancer cells treated with OEF and without frequency modulation continued to increase, whereas none of the breast cancer cells receiving OEF with frequency modulation survived.

Furthermore, we recently performed an experiment on a different line of breast cancer cells at 24 hours wherein we tested the cells for apoptosis (regarded as a form of “programmed cell death”) in an attempt to further understand why cells were dying from the treatment. As shown by the results in TABLE 1 herewith, we are seeing much greater treatment effect with frequency modulation than without. This could prove to be a major advance in treatment, broadly speaking, particularly as results are duplicated and for particular cancer cells, the frequency selection and modulation patterns are adjusted to maximize the delivery of frequency modulation (treatment) with the presently disclosed subject matter.

TABLE 1

| | Quantified Apoptosis in | | | |
|---|---|---|---|---|
| | HCC38 Cells after 24 Hours | | | |
| | | Early | Late | |
| | Live | Apoptosis | Apoptosis | Dead |
| Control | 88.04% | 10.53% | 1.43% | 0% |
| No Modulation | 65.98% | 34.02% | 0% | 0% |
| Frequency Modulation | 20.56% | 79.44% | 0% | 0% |

n = 3

It should also be understood that, in the broader context, “treatment” herewith includes and encompasses use of the presently disclosed technology in any (and all) of the three phases of testing (or use) with cell culture plated known cancer cells (in vitro methodology), testing in animals, and therapeutic and/or testing uses with humans. In other words, a “target volume” for “treatment” within the presently disclosed subject matter may relate to one or more cell cultures, a volume of cells within test animals, or a volume of cells with human patients.

Similarly, presently disclosed subject matter may relate to treatment protocols applicable outside strictly the area of disease referred to as “cancer.” For example, it would be expected that presently disclosed OEF would also prove a successful treatment for tissue fibrosis. There are many causes of tissue fibrosis (e.g., infection, injury, autoimmune, radiation, etc.) which can lead to pathologic disease (e.g., spinal cord fibrosis and permanent paralysis; post-ARDS (Acute Respiratory Distress Syndrome) lung fibrosis, which can be fatal; post-COVID-19 lung fibrosis; and radiation fibrosis, which often can be quite morbid and limits doses of radiation that can be safely delivered, etc.). Based on current knowledge, we believe that OEF, if delivered at the time of insult, may successfully limit the degree to which tissue fibrosis occurs. Effective treatment protocols may exist for use in connection with other disease processes.

Still further, various exemplary embodiments of presently disclosed subject matter may relate to various frequency practices, including, in some instances, use of amplitude modulation of OEF. One example of the benefit of amplitude modulation of OEF with a signal frequency less than 1 KHz is the ability to achieve a system capable of delivering forced oscillation of the dielectric force exerted upon cells when undergoing mitosis. Forced oscillations occur when an oscillating system is driven by a periodic force that is external to the oscillating system^{[10]}. In such example, the oscillating system becomes the displacement (by the dielectric force of the DNA strands and/or the septin complexes involved in the assembly of the microtubules) from their natural movement that occurs during mitosis or any other process involving the assembly of the cytoskeleton. By modulating the amplitude of the OEF delivered to the cells with a signal frequency less than 1 KHz, this displacement within the created oscillating system occurs.

When a driving force is applied to an oscillating system at a frequency near the natural frequency of the system, the amplitude of the oscillation can become quite large^{[10]}. Therefore, the use of amplitude modulation as presently disclosed has the potential to cause the magnitude of the dielectric force to be much greater, resulting in far more catastrophic intracellular damage to cells during mitosis than if the OEF are delivered without amplitude modulation. By analogy, although likely far more complex (such as potentially also involving resonance issues), a common example of this principle of forced oscillation is the failure of the “Galloping Gertie” bridge spanning the Tacoma Narrows strait which dramatically collapsed on Jul. 1, 1940[¹¹].

While various ranges of frequencies have been discussed herein, further presently disclosed examples involve yet other arrangements. For example, we have used our presently disclosed device to test the effect of amplitude modulation with a signal frequency of 8 MHz and an OEF carrier frequency of 150 kHz, which was delivered to cultures of MDA-MB-231 TNBC (triple negative breast cancer) cells. In one such experiment we conducted, no tumor cells survived after 72 hours of exposure (i.e., treatment per presently disclosed apparatus and methodology). Such same degree of cell death did not occur when OEF were delivered by the presently disclosed device but without use of amplitude modulation. Since the presently disclosed device is capable of generating signals of much lower frequencies than 1 kHz and of arbitrary shape, it is capable of testing and delivering OEF to create forced oscillation of the dielectric force that can be tuned to maximize the intracellular destruction within targeted volumes whether delivered to tissue culture, animals or human patients.

Other presently disclosed exemplary embodiments relate to examples of using much higher frequencies, such as in the range of 1 MHz or greater, to shape the electric field distribution three dimensionally through the use of multiple electrodes. In some embodiments, that may preferably include five or more electrodes.

The following is an example of the 3D shaping of the OEF that would require alternating the distribution of OEF to electrode pairs in a given frequency range. However, the frequency intended is not a carrier frequency. Per presently disclosed subject matter, the OEF are not “carried” at the frequency but rather switched between pairs of electrodes at the frequency, which is quite different than carrier frequency practice. In some such embodiments, in order to have desired arrangements of paired electrodes, a relatively higher number of electrodes may be desired (e.g., six or more electrodes). In some other embodiments, it would be possible to use a multitude of electrodes (independent electrodes). For example, in some such independent electrode arrangements, an embodiment might use a minimum of six independent electrodes or more, each receiving individual but coordinated signals.

One example of expected benefit of shaping the OEF in three dimensions to a target volume is the ability to reduce the current delivered to tissue or cells outside a target volume such as skin to reduce the skin damage that can be currently experienced with the delivery of TTF^{[12]}. When an oscillating current is applied to a pair of electrodes that are inserted in tissue culture or applied to the skin of an animal or human patient, the charge moves through the target volume from one electrode to the other. The frequency at which these charges move through the target tissue, i.e., signal frequency, is dependent on the frequency of the current applied to the electrode pair. The target volume can conceptually be divided into non-overlapping and continuous arbitrarily small volumes called voxels. If one wanted to 3D shape the intensity of the movement of charge through any one voxel of the target at the same signal frequency, one could deliver current to an array of pairs of smaller electrodes between which the voxel was positioned but would have to alternate between pairs at a far higher frequency than the OEF. This would result in far less current being delivered to tissue outside the intended voxel but between the two larger electrodes. By switching between these electrode pairs at such a high frequency as presently disclosed, the OEF can be shaped to multiple voxels within the target volume. By way of analogy, the 3D shaping of the OEF distribution is similar to the 3D shaping of radiation dose distributions with intensity modulated radiation therapy except that the present disclosed subject matter achieves the desired shaping by the extremely rapid switching of OEF between electrode pairs rather than the rotational movement of a linear accelerator.

With the presently disclosed subject matter, when intending to deliver OEF shaped in three dimensions in order to minimize the amount of current delivered to tissues outside the target area, e.g., skin, one may deliver OEF with a minimum of three pairs of electrodes and a frequency of alternation between pairs of at least 1 MHz and likely much higher.

This written description uses examples to disclose the presently disclosed subject matter, including the best mode, and also to enable any person skilled in the art to practice the presently disclosed subject matter, including making and using any devices or systems and performing any incorporated methods. The patentable scope of the presently disclosed subject matter is defined by the claims, and may include other examples that occur to those skilled in the art. Such other examples are intended to be within the scope of the claims if they include structural and/or step elements that do not differ from the literal language of the claims, or if they include equivalent structural and/or elements with insubstantial differences from the literal languages of the claims.

### REFERENCES

1. Berkelmann, L. et al. Tumour-treating fields (TTF): Investigations on the mechanism of action by electromagnetic exposure of cells in telophase/cytokinesis. Sci. Rep. 9, 7362 (2019).
2. Jo, Y. et al. Selective toxicity of tumor treating fields to melanoma: an in vitro and in vivo study. Cell Death Discov. 4, 46 (2018).
3. Kirson, E. D. Disruption of Cancer Cell Replication by Alternating Electric Fields. Cancer Res. 64, 3288-3295 (2004).
4. Kirson, E. D. et al. Alternating electric fields (TTF) inhibit metastatic spread of solid tumors to the lungs. Clin. Exp. Metastasis 26, 633-640 (2009).
5. Giladi, M. et al. Overcoming Cell Size Escape From Tumor Treating Fields Using A Varying Frequency Treatment Paradigm In-Vitro, Rosa Schneiderman, Mijal Munster, Uri Weinberg, Eilon Kirson, and Yoram Palti. Neuro-Oncol. 15, iii43 (2013).
6. Gera, N. et al. Tumor Treating Fields Perturb the Localization of Septins and Cause Aberrant Mitotic Exit. PLOS ONE 10, e0125269 (2015).
7. Swanson, K. D., Lok, E. & Wong, E. T. An Overview of Alternating Electric Fields Therapy (NovoTTF Therapy) for the Treatment of Malignant Glioma. Curr. Neurol. Neurosci. Rep. 16, (2016).
8. https://www.zylotherapeutics.com/
9. Chen C, Jensen B K, Mistry G, Wyss R, Zultak M, Patel I H, Rakhit A K. Negligible systemic absorption of topical isotretinoin cream: implications for teratogenicity. J Clin Pharmacol. 1997 April; 37(4):279-84. doi: 10.1002/j. 1552-4604.1997.tb04303.x. PMID: 9115052.
10. Stanford, A. L. & Tanner, J. M. Oscillations. in Physics for Students of Science and Engineering 210-233 (Elsevier, 1985). doi:10.1016/B978-0-12-663380-1.50011-2.
11. Billah, K. Y. & Scanlan, R. H. Resonance, Tacoma Narrows bridge failure, and undergraduate physics textbooks. American Journal of Physics 59, 118-124 (1991).
12. Lacouture, M. E. et al. Prevention and Management of Dermatologic Adverse Events Associated With Tumor Treating Fields in Patients With Glioblastoma. Front Oncol 10, 1045 (2020).

What is claimed is:

1. Methodology for using oscillating electric fields (OEF) for passing through a target volume as treatment:
selectively and controllably generating an oscillating electric signal at least one output thereof;
connecting the output to an electrode associated with a target volume;
controlling generation of the oscillating electric signal such that the electric field strength output at the electrode is in a range of from 0 up to 10 V/cm and such that the electric field passes through the target volume; and
changing at least one of either the frequency or amplitude of the oscillating electric signal at least once during a treatment.

2. Methodology according to claim 1, wherein the target volume comprises a volume of cells within at least one of A one or more cell cultures, B test animals, or C human patients.

3. Methodology according to claim 2, further comprising a plurality of outputs and corresponding plurality of electrodes.

4. Methodology according to claim 3, wherein the target volume comprises a volume of cells within a human patient, and the electrodes are positioned for OEF to pass through at least one targeted anatomical volume of the patient.

5. Methodology according to claim 4, wherein the anatomical volume comprises a patient's brain, lungs, liver, pancreas, abdomen, pelvis, or other anatomical organ or region of the body.

6. Methodology according to claim 2, wherein the electric field strength output at the electrode is in a range of from 2 V/cm up to 10 V/cm.

7. Methodology according to claim 2, further including:
modulating the frequency of the oscillating electric signal over a range of frequencies; and
varying the peak electric field strengths.

8. Methodology according to claim 3, wherein the electrodes comprise an array of relatively smaller, greater in number electrodes to which oscillating electric signals are delivered individually and at relatively higher frequencies up to GHz range.

9. Methodology according to claim 8, further comprising oscillating delivery of current between individual arrangements of electrodes to treat portions of a target volume in accordance with determined custom 3-dimensional shaping of electric field distributions.

10. Methodology according to claim 3, further including real-time cooling of the electrodes during OEF delivery to reduce risk of thermal damage to a patient's skin.

11. Methodology according to claim 2, further comprising:
prior to application of OEF, synchronizing mitotic cycle of targeted cancer cells through small radiation doses or chemical blockers; and
coordinating cell cycle with amplitude modulation, frequency modulation, or commutation time of the oscillating electric signal.

12. Methodology according to claim 2, further comprising, concurrent, sequential, or both, with application of OEF, practicing at least one other therapeutic application including therapeutic radiation in single or multiple fractions, ionizing radiation, chemotherapy, immunotherapy, molecular targeted therapy, nanotherapy, surgery, ultrasound, thermal therapy, hyperthermia, hyperbaric oxygen therapy, angiogenesis inhibitors, and antioxidants.

13. Methodology according to claim 4, further comprising prior to application of OEF,
applying selected cream on skin tissue under locations for electrodes to be placed, to function as a dielectric gel between the electrodes and skin, and to slow skin growth at the site; or
applying selected cream on skin tissue under locations for electrodes to be placed, to penetrate into the skin for altering its electrical impedance toward a more favorable value, minimizing the skin's influence on the electric field's shape and strength.

14. Apparatus for passing oscillating electric fields (OEF) through a target volume as treatment:
a controllable oscillator for generating an oscillating electric signal at least one output thereof;
at least one electrode connectable with the at least one output, associated with a target volume;
a power amplifier for controllably amplifying the oscillating electrical signal at the at least one output, so that the electric field strength output at the electrode when connected to the output is in a range of from 0 up to 10 V/cm, and passes through the target volume; and
one or more processors programmed to control the output level of the power amplifier, and to control and change at least one of either the frequency or amplitude of the oscillating electric signal at least once during a treatment.

15. Apparatus according to claim 14, wherein the target volume comprises a volume of cells within at least one of A one or more cell cultures, B test animals, or C human patients.

16. Apparatus according to claim 15, further comprising a plurality of outputs and corresponding plurality of electrodes.

17. Apparatus according to claim 16, wherein the target volume comprises a volume of cells within a human patient, and the electrodes are positioned for OEF to pass through at least one targeted anatomical volume of the patient.

18. Apparatus according to claim 15, wherein the one or more processors are further programmed to control the output level of the power amplifier for the electric field strength output at the electrode to be in a range of from 2 V/cm up to 10 V/cm.

19. Apparatus according to claim 15, wherein the one or more processors are further programmed for:
modulating the frequency of the oscillating electric signal over a range of frequencies; and
varying the peak electric field strengths.

20. Apparatus according to claim 16, wherein the electrodes comprise an array of relatively smaller, greater in number electrodes to which oscillating electric signals are delivered individually, and wherein the one or more processors are further programmed to control the frequency of the oscillating electric signal to be at relatively higher frequencies up to GHz range.

21. Apparatus according to claim 15, wherein the one or more processors are further programmed for coordinating at least one of amplitude modulation, frequency modulation, or commutation time of the oscillating electric signal with cell cycle.

22. Methodology for using Tumor Treating Fields (TTF) for cancer treatment by passing oscillating electric fields (OEF) through a targeted anatomical volume of a patient, to use disrupting a targeted cell's ability to divide as disease treatment, comprising:
providing a controllable oscillating electric signal generator, connected to a plurality of electrodes associated with a patient, with the output of the electrodes limited for electric field strength up to 10 V/cm; and
controlling at least one of either the frequency or amplitude of the oscillating electric signal to be varied during the course of a treatment.

23. Methodology according to claim 22, further comprising controlling both the frequency and amplitude of the oscillating electric signal to be varied during the course of a treatment, with the electric field strength being varied in a range of from 2 V/cm up to 10 V/cm.

24. Methodology according to claim 23, wherein the targeted anatomical volume comprises a patient's brain, lungs, liver, pancreas, abdomen, pelvis, or other anatomical organ or region of the body.

25. Methodology according to claim 22, wherein the electrodes comprise an array of relatively smaller, greater in number electrodes to which oscillating electric signals are delivered individually and at relatively higher frequencies up to GHz range.

26. Methodology according to claim 25, further comprising oscillating delivery of current between individual arrangements of electrodes to treat portions of a targeted anatomical volume in accordance with determined custom 3-dimensional shaping of electric field distributions.

27. Methodology according to claim 22, further comprising prior to application of OEF:
synchronizing mitotic cycle of targeted cancer cells within a targeted anatomical volume through small radiation doses or chemical blockers, and coordinating cell cycle with amplitude modulation, frequency modulation, or commutation time of the oscillating electric signal; or
applying selected cream on skin tissue under locations for electrodes to be placed.

28. Methodology according to claim 22, further comprising, concurrent, sequential, or both, with application of OEF, practicing at least one other therapeutic application including therapeutic radiation in single or multiple fractions, ionizing radiation, chemotherapy, immunotherapy, molecular targeted therapy, nanotherapy, surgery, ultrasound, thermal therapy, hyperthermia, hyperbaric oxygen therapy, angiogenesis inhibitors, and antioxidants.

29. Methodology according to claim 22, further comprising varying at least one of: A the frequency of the oscillating electric signal over a range of frequencies, or B the electric field strength output at the electrode over a range of voltages, wherein varying is varied according to a predetermined profile.

30. Methodology according to claim 29, wherein the predetermined profile comprises at least one of a mathematical function and an arbitrary signal shape.

31. Methodology according to claim 30, wherein the mathematical function comprises at least one of a sawtooth, triangle, sinusoidal, sin\hat{ }2, or bimodal function.

32. Methodology according to claim 30, wherein the predetermined profile comprises a central frequency or amplitude or multiple peaks, each with a maximum deviation, or a weighted deviation in order to increase time at specific values or range of values.

33. Methodology according to claim 22, further comprising varying at least one of: A the frequency of the oscillating electric signal over a range of frequencies, and B the electric field strength output at the electrode over a range, wherein varying is varied according to feedback, in order to dynamically control the frequency or electric field strength.

34. Methodology according to claim 33, wherein the feedback comprises impedance sensed through the electrode or a plurality of electrodes associated with the patient.

35. Apparatus for passing oscillating electric fields (OEF) through a targeted anatomical volume of a patient as disease treatment:
a controllable oscillator for generating an oscillating electric signal at least one output thereof;
at least one electrode connectable with the at least one output, to be associated with a patient;
a power amplifier for controllably amplifying the oscillating electrical signal at the at least one output, so that the electric field strength output at the electrode when connected to the output is in a range of from 0 up to 10 V/cm; and
one or more processors programmed to control the output level of the power amplifier, and to control and change at least one of either the frequency or amplitude of the oscillating electric signal at least once during a patient treatment.

36. Apparatus according to claim 35, wherein the one or more processors are further programmed to modulate the frequency of the oscillating electric signal over a range of frequencies up to GHz range, and programmed to vary the electric field strength output at the electrode in a range of from 2 V/cm up to 10 V/cm.

37. Apparatus according to claim 35, further comprising an array of relatively smaller, plurality of electrodes to which oscillating electric signals are delivered individually.

38. Apparatus according to claim 35, further comprising a plurality of outputs and corresponding plurality of electrodes associated with a patient.

39. Apparatus according to claim 38, wherein the one or more processors are further programmed to oscillate delivery of current between individual arrangements of electrodes to treat portions of a target volume in accordance with determined custom 3-dimensional shaping of electric field distributions.

40. Apparatus according to claim 39, further comprising selected cream applied on skin tissue under locations for electrodes to be placed, to function as a dielectric gel between the electrodes and skin.

41. Apparatus according to claim 35, wherein the one or more processors are further programmed to at least one of vary: A the frequency of the oscillating electric signal over a range of frequencies, and B the electric field strength output at the electrode over a range of voltages, wherein varying the frequency or the electric field strength is varied according to a predetermined profile.

42. Apparatus according to claim 41, wherein the predetermined profile comprises one of a mathematical function and an arbitrary signal shape.

43. Apparatus according to claim 42, wherein the mathematical function comprises at least one of a sawtooth, triangle, sinusoidal, sin\hat{ }2, or bimodal function.

44. Apparatus according to claim 42, wherein the predetermined profile comprises a central frequency or amplitude or multiple peaks, each with a maximum deviation, or a weighted deviation in order to increase time at specific values or range of values.

45. Apparatus according to claim 35, wherein the one or more processors are further programmed to at least one of vary: A the frequency of the oscillating electric signal over a range of frequencies, and B the electric field strength output at the electrode over a range, wherein varying the frequency or the electric field strength is varied according to feedback to dynamically control the frequency or electric field strength.

46. Apparatus according to claim 43, wherein the feedback comprises impedance sensed through the electrode or a plurality of electrodes associated with the patient.
